(19) United States
(12) Patent Application Publication
(10) Pub. No.: US 2026/0191518 A1
(43) Pub. Date: Jul. 9, 2026

(54) BIOPSY CAVITY IRRIGATION

(71) Applicant: Devicor Medical Products, Inc., Cincinnati, OH (US)

(72) Inventors: Andrew P. NOCK, Dayton, OH (US); Maria B. ROMA, Cincinnati, OH (US); Garrett A. HOUSEHOLDER, Cincinnati, OH (US)

(21) Appl. No.: 19/556,398

(22) Filed: Mar. 4, 2026

Related U.S. Application Data
(63) Continuation of application No. PCT/US2024/048114, filed on Sep. 24, 2024.
(60) Provisional application No. 63/540,964, filed on Sep. 28, 2023.

(51) Int. Cl. A61B 10/04; A61B 10/02; A61M 25/00

(52) U.S. Cl. CPC A61B 10/04; A61B 10/0275; A61M 25/003; A61B 2010/045; A61M 2025/0031

(57) ABSTRACT

A needle for use with a biopsy device includes a cannula and a tissue piercing tip. The cannula defines a cutter lumen configured to receive a cutter of the biopsy device and a lateral lumen proximate the cutter lumen. The tissue piercing tip includes a body defining a plurality of cutting surfaces, a lower opening, and an upper opening. At least the upper opening being defined by a single cutting surface of the plurality of cutting surfaces. The upper opening is in communication with the cutter lumen. The lower opening is in communication with the lateral lumen.

### PRIORITY

This application claims priority to U.S. Provisional Application No. 63/540,964, entitled “Biopsy Cavity Irrigation,” filed on Sep. 28, 2023, the disclosure of which is hereby incorporated by reference herein.

### BACKGROUND

A number of patients will have breast biopsies because of irregular mammograms and palpable abnormalities. Biopsies can include surgical excisional biopsies and stereotactic and ultrasound guided needle breast biopsies. In the case of image directed biopsy, the radiologist or other physician may take a small sample of the irregular tissue for laboratory analysis. If the biopsy proves to be malignant, additional surgery (e.g., a lumpectomy or a mastectomy) may be required. In the case of needle biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers used after breast biopsies to mark the location where the biopsied tissue was removed is described in the following US Patents: U.S. Pat. No. 6,083,524, “Polymerizable biodegradable polymers including carbonate or dioxanone linkages,” issued Jul. 4, 2000; U.S. Pat. No. 6,162,241, “Hemostatic tissue sealants,” issued Dec. 4, 2000; U.S. Pat. No. 6,270,464, “Biopsy localization method and device,” issued Aug. 7, 2001; U.S. Pat. No. 6,356,782, “Subcutaneous cavity marking device and method,” issued Mar. 12, 2002; U.S. Pat. No. 6,605,294, “Methods of using in situ hydration of hydrogel articles for sealing or augmentation of tissue or vessels,” issued Aug. 12, 2003; U.S. Pat. No. 8,600,481, “Subcutaneous cavity marking device,” issued Dec. 3, 2013 and U.S. Pat. No. 8,939,910, “Method for enhancing ultrasound visibility of hyperechoic materials”, issued Jan. 27, 2015. All of these US Patents are incorporated by reference in their entirety.

In some circumstances, saline is used to irrigate the biopsy cavity. In some instances this may be done by, for example, injecting saline through a space between the inner surface of the needle and the outer surface of the cutter. The injected saline is then removed by vacuum communicated through a hollow interior of the cutter. However, some existing methods for saline irrigation may have insufficient removal of saline, which can lead to accumulation of saline within the biopsy cavity. Thus, it may be desirable to provide saline irrigation to a biopsy cavity with improved fluid flow characteristics. While several systems and methods have been made and used for irrigating a biopsy cavity, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an illustrative biopsy needle;

FIG. 2 depicts a side cross-sectional view of the biopsy needle of FIG. 1, the cross-section taken along line 2-2;

FIG. 3 depicts a perspective view of a tissue piercing tip of the biopsy needle of FIG. 1;

FIG. 4 depicts another perspective view of the tissue piercing tip of FIG. 3;

FIG. 5 depicts a perspective cross-sectional view of a tissue piercing tip of FIG. 3, the cross-section taken along line 5-5 of FIG. 3;

FIG. 6 depicts another perspective cross-sectional view of a tissue piercing tip of FIG. 3, the cross-section taken along line 6-6 of FIG. 3;

FIG. 7 depicts a perspective view of the biopsy needle of FIG. 1 disposed in tissue to apply a fluid to a biopsy cavity;

FIG. 8 depict a side cross-sectional view of the biopsy needle of FIG. 1 disposed in tissue to apply a fluid to a biopsy cavity;

FIG. 9 depicts a perspective view of an illustrative marker delivery device for use with the biopsy needle of FIG. 1;

FIG. 10 depicts a detailed perspective view of a cannula of the marker delivery device of FIG. 9;

FIG. 11 depicts a detailed cross-sectional view of the cannula of FIG. 10, the cross-section taken along line 11-11 of FIG. 10;

FIG. 12A depicts a side cross-sectional view of the biopsy needle of FIG. 1 and the cannula of FIG. 10, with the cannula inserted therein;

FIG. 12B depicts another side cross-sectional view of the biopsy needle of FIG. 1 and the cannula of FIG. 10, with the marker delivery device in an intermediate position; and

FIG. 12C depicts yet another side cross-sectional view of the biopsy needle of FIG. 1 and the cannula of FIG. 10, with a marker being deployed through the biopsy needle via the marker delivery device;

FIG. 13 depicts an exploded perspective view of an illustrative alternative tissue piercing tip incorporated into the biopsy needle of FIG. 1; and

FIG. 14 depicts a side cross-sectional view of the piercing tip of FIG. 13 incorporated into the biopsy needle of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Illustrative Biopsy System with Cavity Irrigation System

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification.

As a marker is being positioned at a biopsy site, it may be desirable for the biopsy device to have a fluid source that can be accessed to irrigate the biopsy cavity. For instance, it may be desirable for the needle tip of the biopsy device to include an opening for saline or other fluid to flow through and irrigate the biopsy cavity. This opening may be oriented away from a lateral aperture, thus allowing saline to flow downwardly out of the opening and then upwardly within the biopsy cavity into the lateral aperture. There, a cutter may be utilized to vacuum the saline out of the lateral aperture. Such an irrigation system results in an increased average fluid weight per biopsy by preventing the saline from being vacuumed out before it can accumulate in the biopsy cavity.

In some embodiments, it may be desirable for the saline or other fluid media to be above room temperature, such as above 37 degrees C., when irrigating the biopsy cavity. Using warm saline or other fluid media to irrigate a biopsy cavity can provide hemostatic properties and reduce the amount of blood lost per minute.

In examples where marker deployment includes an irrigation system, it may be desirable to incorporate features into associated devices to support such marker delivery device configurations. While various examples of suitable devices for a marker delivery device with an irrigation system are described herein, it should be understood that various alternative configurations may be used as will be apparent to those of ordinary skill in the art in view of the teachings herein.

A. Illustrative Biopsy Needle with Cavity Irrigation System

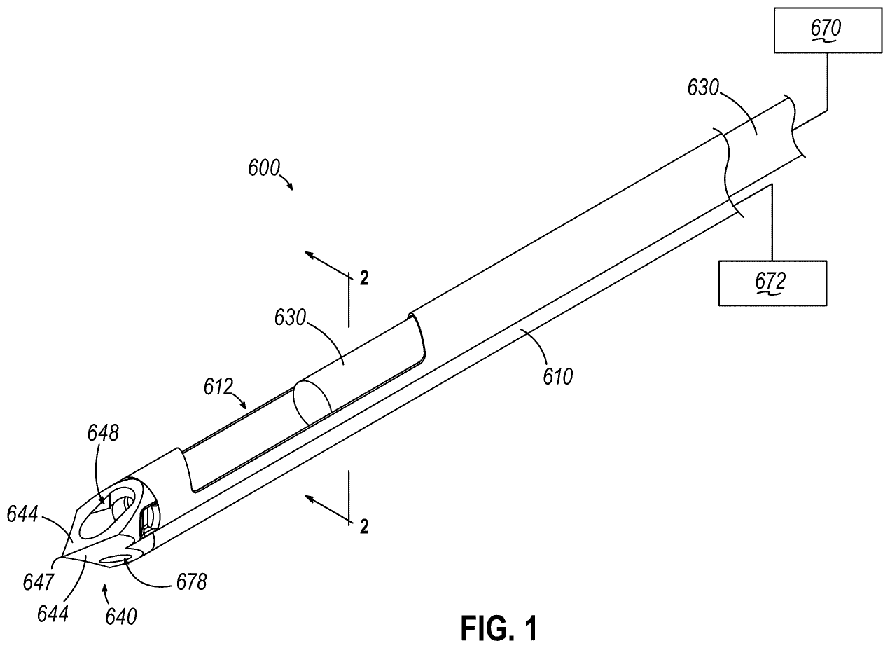

FIG. 1 shows an illustrative biopsy needle (600) that may be readily incorporated into a biopsy device and readily used with various marker delivery devices described herein. Although not shown, it should be understood that in some examples a suitable biopsy device may be configured in accordance with one or more of the teachings of U.S. Pat. No. 9,724,074, entitled “Biopsy Device with Translating Valve Assembly,” issued on Aug. 8, 2017; U.S. Pat. No. 10,799,222, entitled “Apparatus to Allow Biopsy Sample Visualization During Tissue Removal,” issued on Oct. 13, 2020; and U.S. Pat. No. 11,179,141, entitled “Biopsy System,” issued on Nov. 23, 2023, the disclosures of which are hereby incorporated by reference herein. Biopsy needle (600) of the present example comprises an outer cannula (610), a lateral aperture (612) formed therein, and a tissue piercing tip (640) secured to the distal end of outer cannula (610). Although not shown, it should be understood that the proximal end of outer cannula (610) can be coupled to the distal end of a biopsy device (not shown) to promote the collection of tissue samples via biopsy needle (600).

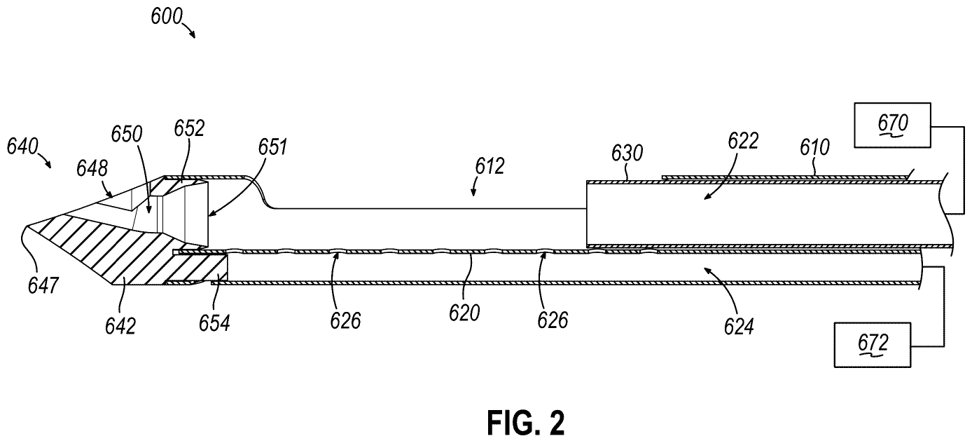

As best seen in FIG. 2, outer cannula (610) includes a cutter receiving tube (620) extending through at least a distal portion of outer cannula (610). Cutter receiving tube (620) defines lateral aperture (612) and is generally circular, while outer cannula (610) is oval-shaped. This configuration provides a two-lumen configuration, with an upper cutter lumen (622) and a lower lateral lumen (624). Both cutter lumen (622) and lower lumen (624) are in fluid communication by one or more fluid openings (626) disposed within a portion of cutter receiving tube (620) opposite of lateral aperture (612).

Cutter receiving tube (620) is generally configured to receive a tubular cutter (630). It should be understood that cutter (630) is generally configured to be movable within cutter receiving tube (620) relative to lateral aperture (612). In some examples, cutter (630) is moved proximally of lateral aperture to “open” lateral aperture (612). Tissue samples are then drawn into lateral aperture (612) by vacuum supplied though cutter lumen (622), lateral lumen (624), or both. Cutter (630) is then advanced distally to sever a tissue sample. The severed tissue sample can then be transported though cutter (630) and into a tissue sample holder or other tissue receiving feature under vacuum supplied by the biopsy device.

Biopsy needle (600) of the present example is in communication with one or more fluid sources (670, 672). As best seen in FIG. 2, cutter (630) is in communication with an axial fluid source (670), while lateral lumen (624) is in communication with a lateral fluid source (672). Both fluid sources (670, 672) are generally configured to communicate a variety of fluids at both positive and negative pressures. For instance, in some examples, fluid sources (670, 672) are configured to selectively communicate fluids such as vacuum, atmospheric air, saline or other fluids, and/or etc. to cutter (630) and/or lateral lumen (624). As will be described in greater detail below, in some examples communication of fluids via fluid sources (670, 672) is coordinated to supply fluid to one portion of biopsy needle (600), while removing fluid from another portion of biopsy needle (600).

In examples including saline as one of the fluids communicated via fluid sources (670, 672), such saline may additionally include other additives such as pharmaceuticals. In addition, or in the alternative, fluids such as saline are heated in some examples. Such heating may include heating a predetermined temperature above room temperature (e.g., above 20° C. or 68° F.). Suitable predetermined temperatures may include, for example, above 37° C. or 98.6° F. In other examples, suitable predetermined temperatures may correspond to a patient's body temperature.

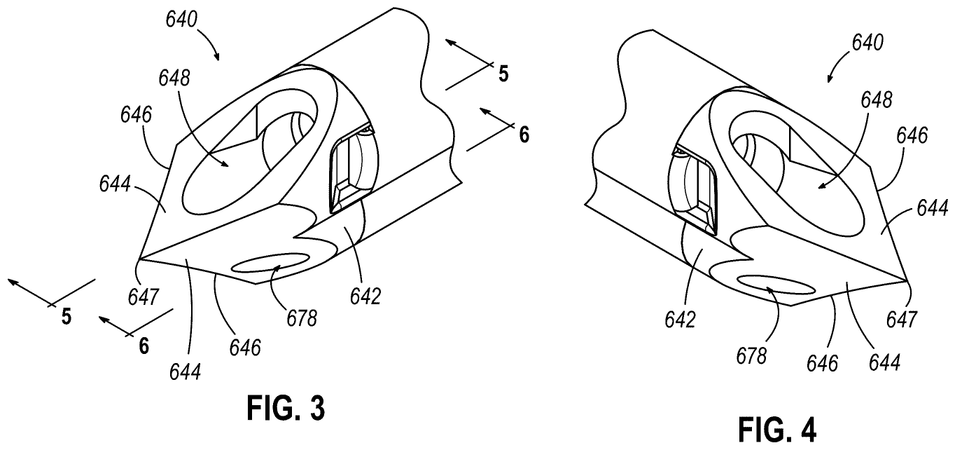

Piercing tip (640) is best shown in FIGS. 3 through 6. As can be seen, piercing tip (640) includes a body (642) defining a plurality of cutting surfaces (644) on the distal end of piercing tip (640). In the present example, three cutting surfaces (644) are arranged around the exterior of body (642) to converge thereby forming a plurality of cutting edges (646). Cutting edges (646) similarly converge to form a sharp distal tip (647). Although surfaces (644) are referred to herein as “cutting surfaces,” it should be understood that actual cutting of tissue can be performed by other structural elements. For instance, in the present example cutting edges (646) and sharp distal tip (647) actually perform cutting. Meanwhile, cutting surfaces (644) merely define the shape and or structure of cutting edges (646) and sharp distal tip (647). Thus, use of the term “cutting” herein in connection with the term “surface” does not necessarily imply that physical cutting of tissue is performed by such cutting surfaces (644).

Cutting surfaces (644) can be formed in the exterior of body (642) in a variety of ways. For instance, in some examples cutting surfaces (644) can be roughly formed by a molding technique such as metal injection molding. Next, cutting surfaces (644) can be further defined by cutting, grinding, abrading, or a combination thereof. In other examples, cutting surfaces (644) are formed entirely cutting, grinding, abrading, or a combination thereof. Still other techniques for forming cutting surfaces (644) will be apparent to those of ordinary skill in the art in view of the teachings herein.

Cutting surfaces (644) in the present example are generally flat. In other words, each cutting surface (644) lies within a single plane oriented at an angle relative to the longitudinal axis of body (642). Although each cutting surface (644) in the present example is shown as being oriented at a particular angle relative to the longitudinal axis of body (642), it should be understood that in other examples various alternative angles can be used as will be apparent to those of ordinary skill in the art in view of the teachings herein.

The proximal end of body (642) includes an upper protrusion (652) and a lower protrusion (654) with both extending proximally from a portion of body (642). Upper protrusion (652) and lower protrusion (654) both define a shape generally complementary to a respective portion of outer cannula (610) and/or cutter receiving tube (620). Additionally, upper protrusion (652) and lower protrusion (654) are laterally offset relative to each other such that upper protrusion (652) and lower protrusion (654) are configured for receipt within cutter lumen (622) and lateral lumen (624), respectively. Thus, upper protrusion (652) defines a larger size relative to the size defined by lower protrusion (654) to complement the size of cutter lumen (622) and lateral lumen (624), respectively.

As described above, both upper protrusion (652) and lower protrusion (654) are configured for receipt within at least a portion of outer cannula (610) and/or cutter receiving tube (620). For instance, as best seen in FIG. 2, upper protrusion (652) is configured for receipt within the distal end of cutter receiving tube (620) while also being inside outer cannula (610). In this configuration, upper protrusion (652) is configured to be received within a portion of cutter (630) to promote severing of tissue by increasing shear forces applied to tissue via cutter (630). Meanwhile, lower protrusion (654) is configured for receipt within the distal end of outer cannula (610) between outer cannula (610) and cutter receiving tube (620). As will be described in greater detail below, lower protrusion (654) is further configured to provide a fluid conduit to direct fluid communication between piercing tip (640) and lateral lumen (624).

In the present example, body (642) of tissue piercing tip (640) is coupled to outer cannula (610) and/or cutter receiving tube (620). In the present example, such coupling is achieved by mechanical fastening. Specifically, crimping a portion of outer cannula (610) and/or cutter receiving tube (620) into one or more recesses disposed on the outer surface of body (642). In other examples, various alternative coupling mechanisms are used in addition to, or in lieu of, mechanical fastening. For instance, in some examples, body (642) is coupled to outer cannula (610) and/or cutter receiving tube (620) by a welding process such as laser, arc, or resistance welding. In other examples, body (642) can be coupled to outer cannula (610) and/or cutter receiving tube (620) in a variety of ways such as by adhesive bonding, other mechanical fastening mechanisms, and/or compression fit.

As best seen in FIGS. 3 through 6, body (642) further defines an upper distal opening (648) and one or more lower distal openings (678), with each distal opening (648, 678) being positioned on a single cutting surface (644). Upper distal opening (648) is generally oval-shaped and is in communication with a lumen (650) extending through body (642), as will be described in greater detail below. In the present example, upper distal opening (648) is positioned on the cutting surface (644) oriented upwardly (e.g., towards the top of the page in FIGS. 3 and 4). However, it should be understood that in other examples, upper distal opening (648) can be positioned on any other cutting surface (644).

Lower distal openings (678) is generally circular-shaped and are in communication with a lumen (680) extending through body (642), as will be described in greater detail below. In the present example, lower distal openings (678) are positioned on one or more cutting surfaces (644) oriented downwardly (e.g., towards the bottom of the page in FIGS. 3 and 4). In other words, lower distal openings (678) are positioned on the cutting surfaces (644) opposite the cutting surface (644) associated with upper distal opening (648). However, it should be understood that in other examples, lower distal openings (678) can be positioned on any other cutting surface (644). Although the present version includes two lower distal openings (678) (one on each side), it should be understood that in other examples, more or less lower distal openings (678) can be used depending on a number of factors such as the number of cutting surfaces (644) and/or the desired volume of fluid flow through lower distal openings (678).

In the present example, distal openings (648, 678) are positioned entirely within a single cutting surface (644) to generally avoid interaction between each distal opening (648, 678) and tissue while biopsy needle (600) is penetrating tissue. In such circumstances, that majority of the force between cutting surfaces (644) and tissue is directed towards cutting edges (646). Thus, as tissue piercing tip (640) penetrates tissue, such tissue will generally move over distal openings (648, 678) rather than snagging or being abraded by distal openings (648, 678).

Although distal openings (648, 678) are described herein as being positioned entirely on a single cutting surface (644), it should be understood that in other examples distal openings (648, 678) may be positioned across multiple cutting surfaces (644). For instance, in some examples, one or more distal openings (648, 678) can be positioned adjacent to distal tip (647) and can thus intersect all three cutting surfaces (644). In such examples, the combination of distal tip (647) and cutting surfaces (644) may be similar to a hypodermic needle.

As will be described in greater detail below, upper distal opening (648) is generally configured to communicate a marker out of body (642). Thus, it should be understood that the size or diameter of upper distal opening (648) generally corresponds to the size or diameter of a marker such as markers (100, 300) described herein. Similarly, since upper distal opening (648) in the present example is positioned entirely on a single cutting surface (644), it should be understood that one or more cutting surfaces (644) can also be sized in proportion to the size or diameter of a marker. While being large enough to accommodate a marker, upper distal opening (648) is also small enough to generally prevent ingress of tissue. As will be described in greater detail below, this sizing permits upper distal opening (648) to self-seal using adjacent tissue when upper distal opening (648) is not in use for marker delivery or other purposes.

As will also be described in greater detail below, lower distal openings (678) are generally configured to communicate fluids relative to body (642). Thus, it should be understood that the size or diameter of lower distal openings (678) is generally sized to facilitate the flow of fluid through lower distal openings (678). Because lower distal openings (678) are positioned entirely on a single cutting surface (644), it should be understood that one or more cutting surfaces (644) can also be sized in proportion to lower distal openings (678). In the present example, the size or diameter of each lower distal opening (678) is generally smaller than the size or diameter of upper distal opening (648). Thus, cutting surfaces (644) associated with lower distal openings (678) may likewise be smaller than the cutting surface (644) associated with upper distal opening (648) in some examples.

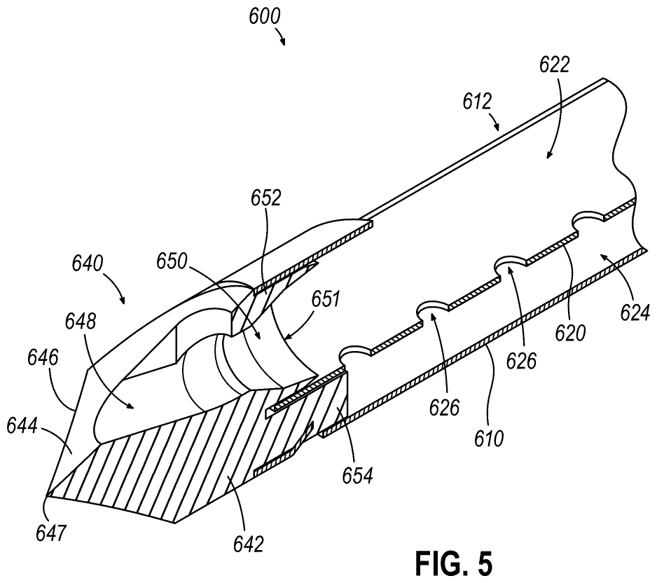

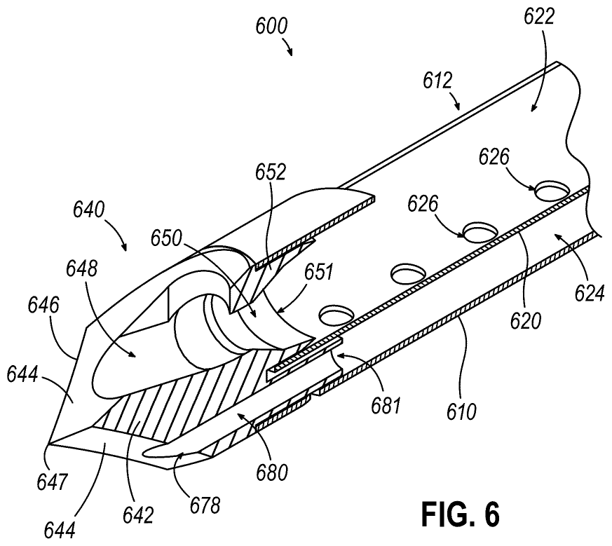

As best seen in FIGS. 5 and 6, body (642) defines lumens (650, 680) extending from upper distal opening (648) and lower distal openings (678), respectively. In particular, upper lumen (650) associated with upper distal opening (648) extends through body (642) to upper proximal opening (651). The cross-sectional diameter or size of lumen (650) generally corresponds to the diameter of upper distal opening (648). Like with upper distal opening (648) described above, upper lumen (650) is generally sized to communicate a marker through body (642) from upper proximal opening (651) to upper distal opening (648). Although the cross-sectional diameter or size of upper lumen (650) of the present example is generally shown as being consistent along the length of body (642), it should be understood that in some examples the cross-sectional diameter or size of upper lumen (650) can vary along the length of body (642). For instance, in some examples the cross-sectional diameter or size of upper lumen (650) can taper inwardly as upper lumen (650) extends distally from upper proximal opening (651). In such examples, upper lumen (650) can thus act as a funnel to direct a marker into a desired position. Of course, other sizes and/or shapes of upper lumen (650) may be apparent to those of ordinary skill in the art in view of the teachings herein.

In some examples, upper lumen (650) is oriented at a slight angle relative to the longitudinal axis of body (642). In such examples, upper lumen (650) is angled such that upper lumen (650) extends upwardly as upper lumen (650) extends distally. Where such an angled orientation is used, this angle is present to account for differences in orientation between upper distal opening (648) and upper proximal opening (651). By way of example only, the specific angle of upper lumen (650) in some examples is approximately 5°. However, it should be understood that in other examples the angle of upper lumen (650) can range between 2 and 10°. In still other examples, various other angles may be used depending on the particular size, shape, and geometry of tissue piercing tip (640).

Lower lumen (680) is associated with each lower distal opening (678). Each lower lumen (680) extends through body (642) to a lower proximal opening (681). The cross-sectional diameter or size of each lower lumen (680) generally corresponds to the diameter of a respective lower distal opening (678). Like with lower distal openings (678) described above, each lower lumen (680) is generally sized to communicate a fluid relative to body (642) from a respective lower proximal opening (681) to a respective lower distal opening (678). Although the cross-sectional diameter or size of each lower lumen (680) of the present example is generally shown as being consistent along the length of body (642), it should be understood that in some examples the cross-sectional diameter or size of each lower lumen (680) can vary along the length of body (642). For instance, in some examples the cross-sectional diameter or size of each lower lumen (680) can taper inwardly as each lower lumen (680) extends distally from a respective lower proximal opening (681). In such examples, each lower lumen (680) can thus act as a funnel to direct fluids into a desired position or fluid flow condition (e.g., laminar or turbulent flow). Of course, other sizes and/or shapes of each lower lumen (680) may be apparent to those of ordinary skill in the art in view of the teachings herein.

Upper proximal opening (651) is defined by upper protrusion (652) of body (642), while lower proximal openings (681) are defined by lower protrusion (654) of body (642). As best seen in FIG. 5, upper proximal opening (651) is in communication with cutter lumen (622) such that upper lumen (650) is configured to communicate with cutter lumen (622) and or cutter (630). Similarly, as best seen in FIG. 6, lower proximal openings (681) are in communication with lateral lumen (624) such that each lower lumen (680) is configured to communicate with lateral lumen (624). As will be described in greater detail below, this configuration permits a marker to be delivered through cutter (630) and into upper lumen (650). As will also be described in greater detail below, this configuration also permits a fluid to be both communicated to a biopsy site via each lower lumen (680) and/or upper lumen (650) and subsequently evacuated from the biopsy suite using each lower lumen (680) and/or upper lumen (650).

### B. Illustrative Irrigation at Biopsy Site

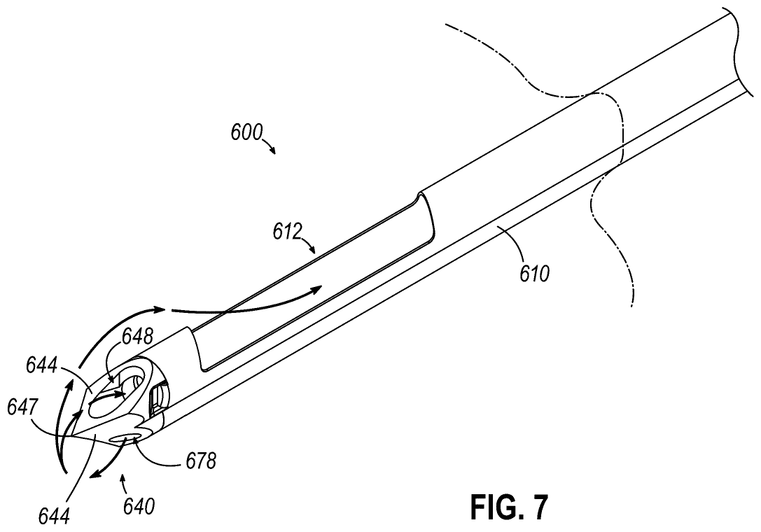

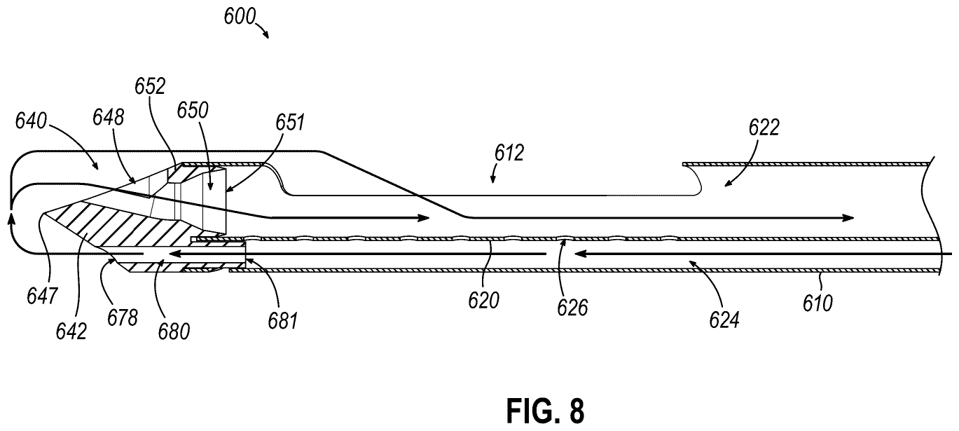

FIGS. 7 and 8 show an illustrative use of biopsy needle (600) described above to irrigate a biopsy site. As best seen in FIG. 7, biopsy needle (600) is inserted into tissue of a patient such that piercing tip (640) and lateral aperture (612) are disposed within the tissue. In some uses, one or more tissue samples may be collected using biopsy needle prior to irrigation to form a biopsy cavity, which may be subsequently irrigated. In addition or in the alternative, irrigation may be performed by to any collection of tissue samples.

Once irrigation of tissue is desired, irrigation is initiated using lateral fluid source (672). In particular, saline and/or other fluids are communicated through lateral lumen (624) via lateral fluid source (672). Such saline and/or other fluids then travel distally though lateral lumen (624), into lower lumen (680) of piercing tip (640), and out lower distal openings (678). Saline and/or other fluids are then communicated into the region surrounding piercing tip (640). Application of saline and/or other fluids can continue until a desired level of irrigation is achieved.

At one or more points during irrigation, it may be desirable to evacuate the saline and/or other fluids from the tissue and/or biopsy cavity. Evacuation of the saline and/or other fluids is performed by communicating a vacuum to cutter (630) via axial fluid source (670). The vacuum is then communicated distally though cutter (630) and into both upper lumen (650) of piercing tip (640) and lateral aperture (612) of biopsy needle (600). In the presence of the vacuum, the saline and/or other fluid is suctioned away from the tissue through upper distal opening (648) of piercing tip (640) and/or lateral aperture (612) of needle (600).

The particular timing of saline and/or other fluids communicated via lateral fluid source (672) and vacuum communicated via axial fluid source (670) may be varied. For instance, in the present example, both saline and/or other fluids are communicated contemporaneously with communication of vacuum. This contemporaneous communication results in a fluid flow from lower distal openings (678) to upper distal opening (648) and/or lateral aperture (612) as best seen in FIG. 8. This particular flow pattern may be desirable to both irrigate tissue, while also limiting accumulation of saline and/or other fluid within the patient. In other uses, the application of saline and/or other fluids is synchronized with the application of vacuum in various ways. For instance, in some uses, the application of vacuum may be delayed relative to the application of saline and/or other fluids to allow at least some accumulation of saline and/or other fluids. In other examples, the application of vacuum is simultaneous or even precedes the application of saline and/or other fluids to minimize the accumulation of saline and/or other fluids.

In the present use, the flow of saline, other fluids, and/or vacuum is substantially continuous. In other uses, the flow of any of saline, other fluids, or vacuum can be pulsed in a variety of ways. For instance, in some uses, vacuum is pulsed while saline and/or other fluids maintain a continuous flow. In other uses, vacuum is continuous while saline and/or other fluids are pulsed. In still other examples, both vacuum and saline and/or other fluids are pulsed.

In the present use, lower distal openings (678) are used as a fluid outlet, while upper distal opening (648) and/or lateral aperture (612) are used as a fluid inlet. This particular configuration may be desirable in some uses to force saline and/or other fluid into a biopsy cavity. In other uses, the configuration is reversed with lower distal openings (678) being used as a fluid inlet and upper distal opening (648) and/or lateral aperture (612) being used as a fluid outlet.

In the present use, the saline and/or other fluids is heated to a predetermined temperature during irrigation. As described above, various suitable predetermined temperatures may be used. For instance, in some uses the predetermined temperature is above room temperature (e.g., above 20° C. or 68° F.). In other uses, the predetermined temperature is above 37° C. or 98.6° F. In yet other uses, the predetermined temperature may correspond to a patient's body temperature. In still other uses, the predetermined temperature is operator adjustable and may be adjusted based on feedback acquired during the procedure. Regardless, in such uses, a relatively warm temperature may be desirable to provide hemostatic properties, therapeutic effects, and reduce the amount of blood lost per minute.

C. Illustrative Marker Delivery Device with End Deployment

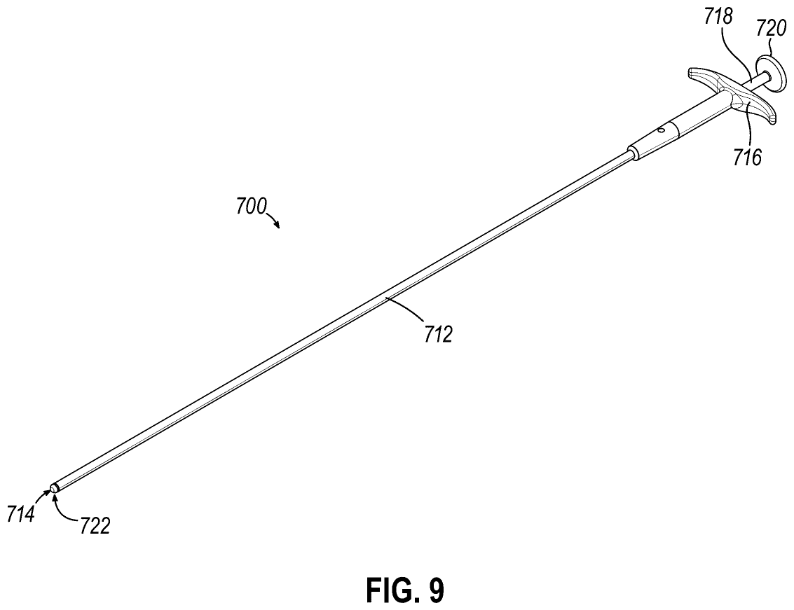

FIGS. 9 through 11 show an illustrative marker delivery device (700) that can be used with needle (600) described above. As best seen in FIG. 8, marker delivery device (700) includes an elongate outer cannula (712) having a marker exit (714). Outer cannula (712) is generally sized to slidably fit within cutter (630) of needle (600). Similarly, the length of cannula (712) is generally sized to correspond to the length of needle (600). It should be understood that in some examples the particular length of cannula (712) can be greater than the length of needle (600). For instance, in some examples the proximal end of needle (600) and/or cutter (630) can be coupled to a tissue sample holder or other tissue collection device. In such examples, the length of cannula (712) can have a length suitable to extend through both needle (600)/cutter (630) and various tissue collection devices.

A grip (716) can be provided at the proximal end of cannula (712). A push rod (718) can be provided, with push rod (718) extending coaxially in cannula (712) such that push rod (718) is configured to translate within cannula (712) to displace one or more markers through marker exit (714). Push rod (718) generally has sufficient rigidity in compression to push a marker from an internal lumen (715) of cannula (712) out through marker exit (714), yet be relatively flexible in bending. A plunger (720) is coupled at the proximal end of push rod (718) for forcing push rod (718) distally in cannula (712) to deploy a marker out of cannula (712).

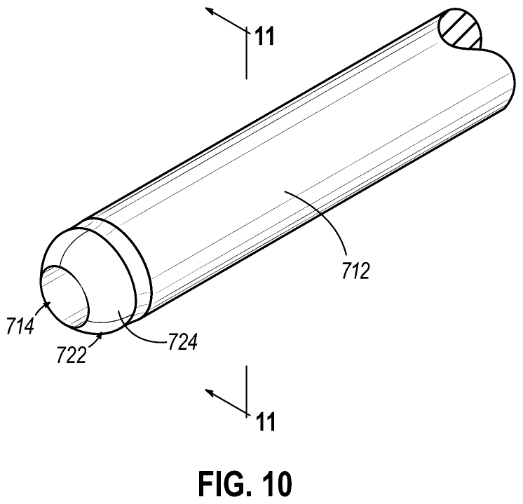

FIG. 10 shows a distal end (722) of cannula (712) in greater detail. As can be seen, marker exit (714) is disposed in the center of distal end (722). Thus, it should be understood that marker exit (714) is generally positioned co-axially with internal lumen (715) of cannula (712). Accordingly, marker delivery device (700) of the present example is an “end-deploy” marker delivery device, rather than a “side-deploy” marker delivery device. As will be described in greater detail below, this configuration generally permits marker delivery device (700) to be used with needle (600) to deploy a marker from upper distal opening (648) of piercing tip (640) rather than lateral aperture (612).

Distal end (722) is generally rounded relative to the outer diameter of cannula (712). Accordingly, it should be understood that distal end (722) generally tapers or rounds inwardly towards marker exit (714). As will be understood, this configuration generally avoids snagging of distal end (722) on various internal geometries of needle (600). Although distal end (722) is shown as being rounded, it should be understood that various other alternative geometries can be used such as frustoconical, tapered, and/or etc.

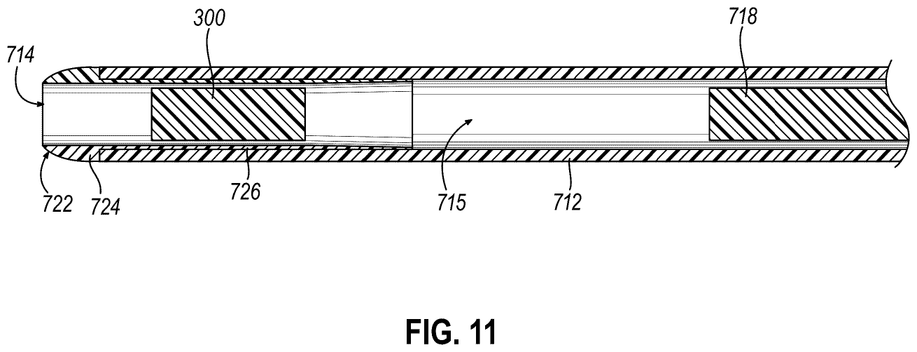

As best seen in FIG. 11, distal end (722) is shown as a separate component inserted into cannula (712). In particular, distal end (722) includes a distal tip (724) and an inert portion (726). Distal tip (724) defines the rounded portion of distal end (722) that is external to cannula (712). Insert portion (726) extends proximally from distal tip (724) and is disposed within cannula (712). Insert portion (726) defines a tapered diameter that is distally slightly oversized relative to the inner diameter of internal lumen (715). As insert portion (726) extends proximally away from distal tip (724), the diameter of insert portion (726) progressively decreases to being approximately equivalent to the inner diameter of internal lumen (715). This configuration generally provides a press fit arrangement between distal end (722) and cannula (712). Additionally, this configuration provides a gradual transition between cannula (712) and distal end (722) to prevent a marker from snagging at the transition. Although a press or mechanical fit is used in the present example, it should be understood that in other examples distal end (722) can be secured to cannula (712) using other fastening methods such as snap fits, adhesive bonding, welding, and/or etc. In still other examples, distal end (722) can be integral with cannula (712).

Marker delivery device (700) in the present example is shown equipped with marker (300). However, it should be understood that in other examples any suitable alternative markers can be used with marker delivery device (700). Marker (300) is initially disposed within cannula (712) distally of a distal end of push rod (718). Accordingly, it should be understood that push rod (718) can be advanced within cannula (712) to push marker (300) from marker exit (714). Although not shown, it should be understood that in some examples internal lumen (715) can be equipped with detents, internal ridges, rubber gaskets, or other similar features to hold marker (300) within cannula (712) until marker (300) is actuated out of marker exit (714) by push rod (718).

D. Illustrative Method for End Deployment of Biopsy Site Marker

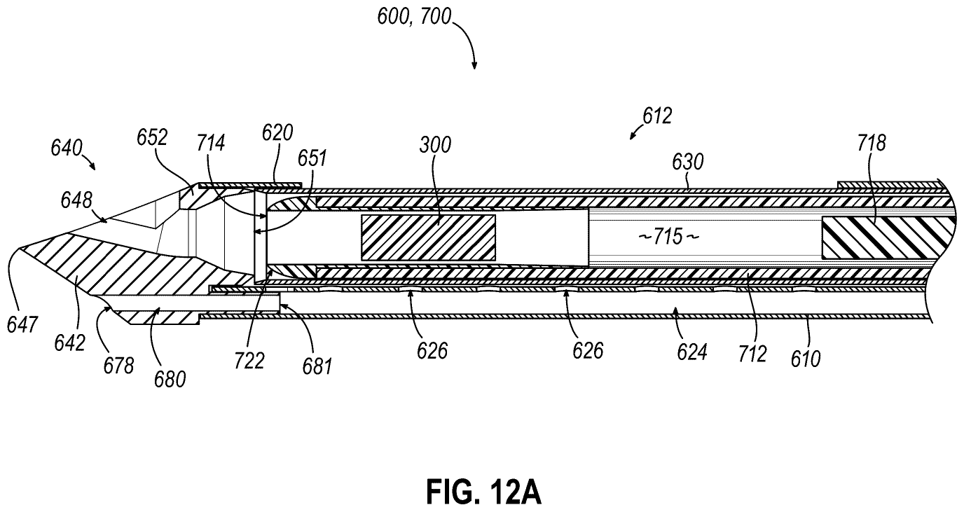

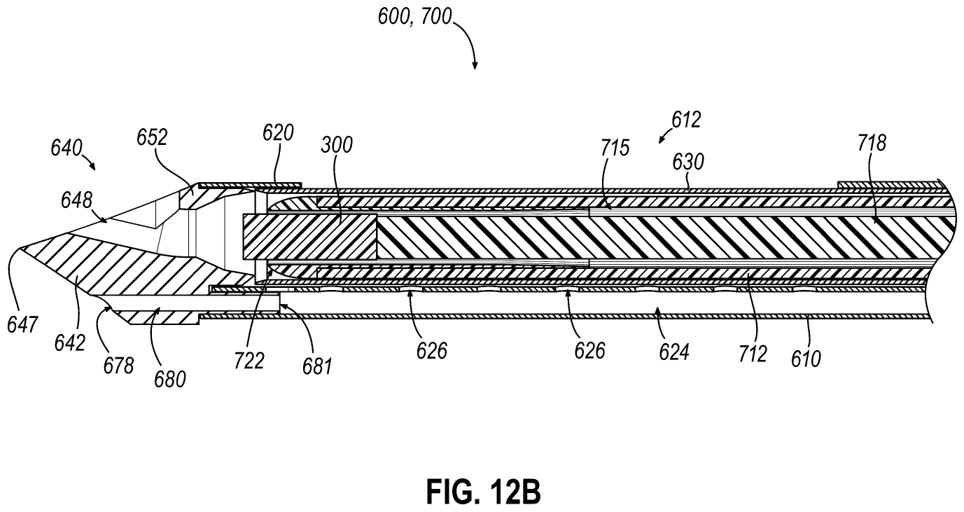

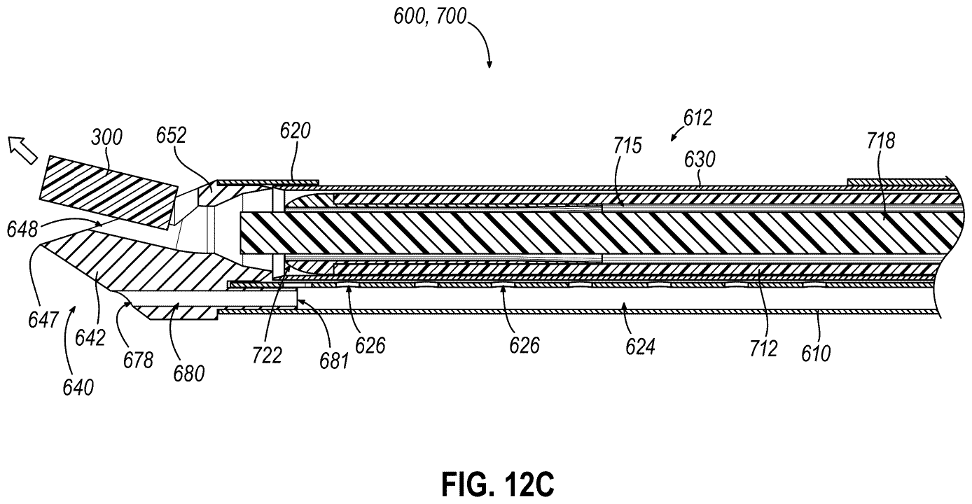

FIGS. 12A through 12C show an illustrative use of marker delivery device (700) with needle (600). As can be seen in FIG. 12A, cannula (712) of marker delivery device (700) is initially inserted into needle (600) through cutter (630). In particular, cannula (712) is inserted into needle (600) through cutter (630) until distal end (722) contacts upper protrusion (652) of piercing tip (640). In this position, cannula (712) is coaxial with cutter (630). This aligns marker exit (714) with upper proximal opening (651) of piercing tip (640).

Once cannula (712) is disposed within needle (600) as shown in FIG. 12A, maker delivery device (700) is positioned for deployment of marker (300). Thus, it should be understood that at this stage marker (300) is disposed within cannula (712). Similarly, push rod (718) is in a pre-actuated state corresponding to a proximal position within cannula (712).

Insertion of marker delivery device (700) into needle (600) for deployment of a marker may occur at a variety of stages during a biopsy procedure. Additionally, marking may be performed before, after, or during the tissue irrigation procedure described above. For instance, in some examples, marker delivery device (700) can be inserted into needle (600) after the completion of a biopsy procedure while needle (600) is still disposed within a patient once all desired tissue samples have been collected from a given biopsy site and all irrigation is completed. Alternatively, marker delivery device (700) can be inserted into needle (600) at one or more intermediate stages during a biopsy procedure. During such intermediate stages, irrigation may be stopped to facilitate marking or may continue. In yet other examples, marker delivery device (700) can be used at other stages where marking of a site of interest is desired. In addition, although not shown, it should be understood that insertion of marker delivery device (700) can be accompanied with other procedural steps such as adjusting a tissue sample holder, removing a tissue sample holder, or removing parts of a tissue sample holder, or removing other portions of a biopsy device.

Regardless of the particular stage of performing marking, it should be understood that in some examples needle (600) may require at least some repositioning relative to the biopsy site prior to marking. For instance, in the present example tissue samples are collected through lateral aperture (612) of needle (600). Meanwhile, marking occurs through upper distal opening (648), which is positioned away from lateral aperture (612). Thus, in some examples it may be desirable to reposition needle (600) to align upper distal opening (648) with the biopsy site that was initially positioned adjacent to lateral aperture (612).

Once cannula (712) is inserted into needle (600) an operator may desire to deploy marker (300) from marker delivery device (700). Deployment of marker (300) is shown in FIGS. 12B and 12C. As can be seen, deployment is initiated by advancing push rod (718) distally using plunger (720) or a combination of plunger (720) and grip (716). As shown in FIG. 12B, advancement of push rod (718) results in the distal end of push rod (718) engaging marker (300) to drive marker distally along with push rod (718).

As marker (300) is driven distally via push rod (718), marker (300) is pushed through internal lumen (715) of cannula (712), through distal end (722), and out of marker exit (714). Once marker (300) is pushed out of marker exit (714), marker enters upper lumen (650) of piercing tip (640) via upper proximal opening (651). Since upper lumen (650) is oriented at an angle as described above, it should be understood that upper lumen (650) can align marker (300) to some extent along the axis of upper lumen (650).

As best seen in FIG. 12C, further advancement of push rod (718) causes marker (300) to proceed further through upper lumen (650) until marker (300) exits needle (600) entirely through upper distal opening (648) in piercing tip (640). Although not shown, it should be understood that in some uses a distal portion of push rod (718) can also exit needle (600) through upper distal opening (648) to provide additional penetration of marker (300) into tissue. However, in other uses push rod (718) may alternatively remain within upper lumen (650) as shown in FIG. 12C.

Once marker (300) is deployed as shown in FIGS. 12A through 12C, marker delivery device (700) can be removed from needle (600). If additional marking is desired, marker delivery device (700) can be reloaded with an additional marker (300). Alternatively, an another identical or substantially identical marker delivery device (700) can be used for deployment of additional markers (300). If no additional marking is desired after deployment of marker (300), needle (600) can also be removed from the patient either in conjunction with removal of marker delivery device (700) or separately.

### II. Illustrative Alternative Piercing Tip

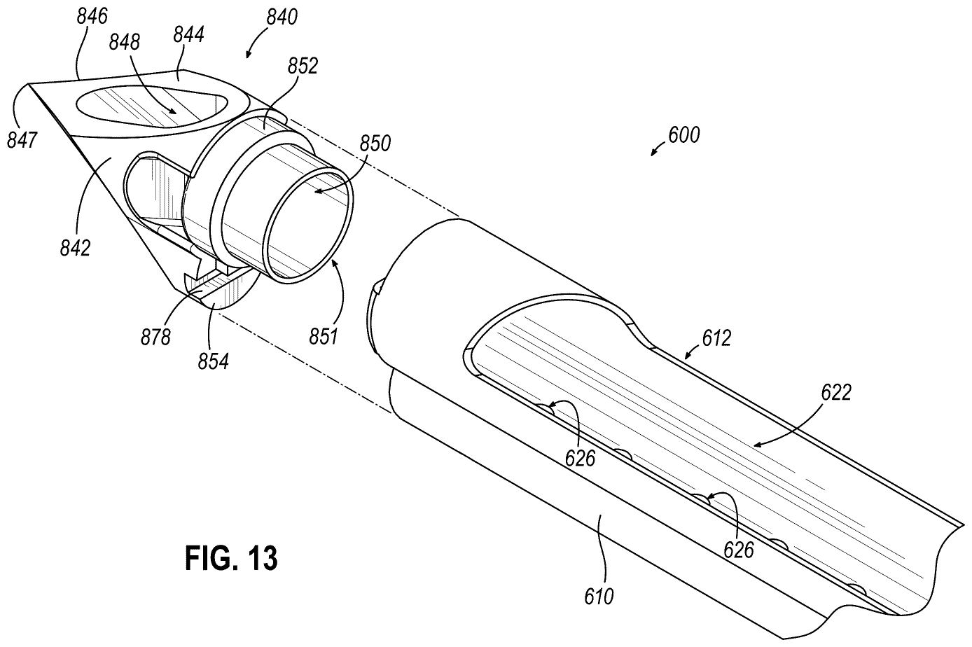

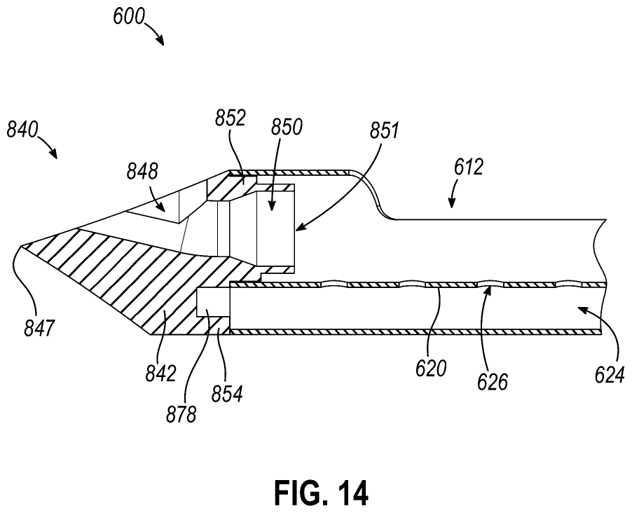

FIGS. 13 and 14 show an illustrative alternative piercing tip (840) that may be readily incorporated into biopsy needle (600) described above in lieu of piercing tip (640). Piercing tip (840) of the present example is substantially similar to piercing tip (640) described above except as otherwise described herein. For instance, like with piercing tip (640) described above, piercing tip (840) of the present example includes a body (842) defining a plurality of cutting surfaces (844) on the distal end of piercing tip (840). As similarly described above, cutting surfaces (844) are arranged around the exterior of body (842) to converge thereby forming a plurality of cutting edges (846), which likewise converge to form a sharp distal tip (847). Cutting surfaces (844) and cutting edges (846) are substantially similar to cutting surfaces (644) and cutting edges (646) described above, such that further details are omitted herein with respect to cutting surfaces (844) and cutting edges (846).

Like body (642) described above, the proximal end of body (842) includes an upper protrusion (852) and a lower protrusion (854) with both extending proximally from a portion of body (842). Upper protrusion (852) and lower protrusion (854) both define a shape generally complementary to a respective portion of outer cannula (610) and/or cutter receiving tube (620). Additionally, upper protrusion (852) and lower protrusion (854) are laterally offset relative to each other such that upper protrusion (852) and lower protrusion (854) are configured for receipt within cutter lumen (622) and lateral lumen (624), respectively. Thus, upper protrusion (852) defines a larger size relative to the size defined by lower protrusion (854) to complement the size of cutter lumen (622) and lateral lumen (624), respectively.

As with body (642) described above, body (842) of the present example further defines an upper distal opening (848) and one or more lower distal openings (878), with each distal opening (848, 878) being positioned on, or proximate, a cutting surface (844). As with upper distal opening (648) described above, upper distal opening (848) is generally oval-shaped and is in communication with a lumen (850) extending through body (842) to a upper proximal opening (851). Similarly, upper distal opening (848) is positioned on the cutting surface (844) oriented upwardly (e.g., towards the top of the page in FIGS. 13 and 14).

Unlike body (642) described above, body (842) of the present example defines a different configuration of lower distal openings (878). In particular, unlike lower distal openings (678) described above, lower distal openings (878) are moved proximally away from cutting surfaces (844) toward lower protrusion (854) on a side of body (842) opposite upper distal opening (848) and upper protrusion (852). Lower distal openings (878) are generally defined by an elongate square or rectangular-shaped passage extending laterally though body (842) from one side to the other. In this configuration, lower distal openings (878) are generally configured to function similarly to lower distal openings (678) described above, but repositioned and reconfigured to provide enhanced ease of manufacturability. Consequently, lower distal openings (878) may be formed together in one process by metal injection molding or a single pass of material removal (e.g., cutting a notch from a portion of lower protrusion (854)).

It should be understood that piercing tip (840) may be used substantially similarly to piercing tip (640) described above. For instance, as similarly described above, upper distal opening (848) is generally configured to facilitate marking via marker delivery device (700). As described above, marker delivery device (700) can be inserted into needle (600). Once marker delivery device (700) is inserted into needle (600), marker delivery device (700) may be aligned with piercing tip (840) to deploy marker (300) through upper distal opening (848). Thus, as with upper distal opening (648) described above, upper distal opening (848) is likewise aligned with cutter (630) and upper cutter lumen (622) of biopsy needle (600). As similarly described above, in some examples upper lumen (850) may be angled or titled relative to the axis of cutter (630) to facilitate marking.

Additionally, piercing tip (840) may be used substantially similarly to piercing tip (640) before or after marker (300) is deployed to irrigate a biopsy site. As described above, saline and/or other fluids may be communicated from fluid source (672) through needle (600), where piercing tip (840) may communicate such fluids. Specifically, saline and/or other fluids may be communicated first out of lower distal openings (878) via lower lateral lumen (624), which permits the saline and/or other fluids to be communicated into the region surrounding piercing tip (840). Once the saline and/or other fluids have accumulated as desired, the saline and/or other fluids can be evacuated using upper distal opening (848) via cutter (630) and/or upper cuter lumen (622). Thus, upper distal opening (848) is in communication with upper cutter lumen (622), while lower distal openings (878) are in communication with lower lateral lumen (624). As described above, in some uses, such communication of fluids and evacuation of fluids may occur contemporaneously, thereby creating a particular flow pattern of fluid around piercing tip (840). As similarly noted above, such a flow pattern may be desirable to irrigate tissue, while also limiting the accumulation of fluids within a patient. Of course, in some uses communication and evacuation of fluids may be synchronized in one or more ways to control the degree of fluid communication proximate piercing tip (840).

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A needle for use with a biopsy device comprising: a cannula defining a cutter lumen configured to receive a cutter of the biopsy device, and a lateral lumen proximate the cutter lumen; and a tissue piercing tip including a body defining a plurality of cutting surfaces, a lower opening, and an upper opening, at least the upper opening being defined by a single cutting surface of the plurality of cutting surfaces, the upper opening being in communication with the cutter lumen, the lower opening being in communication with the lateral lumen.

### EXAMPLE 2

The needle of Example 1, further comprising a lateral aperture, the lower opening being oriented away from a lateral aperture.

### EXAMPLE 3

The needle of Example 2, the upper opening and the lateral aperture both being in communication with the cutter lumen.

### EXAMPLE 4

The needle of any of Example 1 through 3, the upper opening being aligned with the cutter lumen.

### EXAMPLE 5

The needle of any of Examples 1 through 4, the body further defining an upper lumen and a lower lumen, the upper lumen being in communication with the upper opening, the lower lumen being in communication with the lower opening.

### EXAMPLE 6

The needle of Example 5, the cutter lumen defining a cutter axis, the lateral lumen defining a lateral axis, the upper lumen being oriented at an angle with respect to the cutter axis, the lower lumen being aligned with the lateral axis.

### EXAMPLE 7

The needle of any of Example 1 through 6, the cannula including an outer cannula and a cutter receiving tube, the outer cannula and the cutter receiving tube together defining at least a portion of the cutter lumen and the lateral lumen.

### EXAMPLE 8

The needle of Example 7, the cutter receiving tube being disposed between the upper opening and the lower opening.

### EXAMPLE 9

The needle of any of Examples 1 through 8, the body further defining a plurality of

cutting edges, the cutting edges together defining the plurality of cutting surfaces, each of the upper opening and the lower opening being disposed between at least two cutting edges.

### EXAMPLE 10

The needle of Example 9, the plurality of cutting edges converging to form a sharp tip disposed distally of the upper opening and the lower opening.

### EXAMPLE 11

The needle of any of Example 1 through 10, the upper opening being configured to receive a biopsy site marker.

### EXAMPLE 12

The needle of any of Example 1 through 11, the tissue piercing tip being coupled to the cannula by a mechanical fastening mechanism.

### EXAMPLE 13

The needle of any of Examples 1 through 12, the upper opening defining a first shape, the lower opening defining a second shape, the first sample being different from the second shape.

### EXAMPLE 14

The needle of any of Examples 1 through 13, the upper opening defining a first diameter, the lower opening defining a second diameter, the first diameter being greater than the second diameter.

### EXAMPLE 15

The needle of any of Examples 1 through 14, the body further defining a first lower opening and a second lower opening, the first lower opening being oriented on an opposing cutting surface relative to the second lower opening.

### EXAMPLE 16

The needle of any of Examples 1 through 14, the body further defining a first lower opening and a second lower opening, the first lower opening being oriented on an opposing side of the tissue piercing tip, the first lower opening and the second lower opening being offset with respect to the plurality of cutting surfaces.

### EXAMPLE 17

A biopsy system comprising: a biopsy device including a body, a needle extending distally from the body, and a cutter movable relative to the needle, the needle including an outer cannula, a tissue piercing tip secured to a distal end of the outer cannula, a cutter lumen configured to receive the cutter, and a lateral lumen disposed proximate the cutter lumen, the tissue piercing tip defining a lower opening in communication with the lateral lumen and an upper opening in communication with the cutter lumen; a marker delivery device including an elongate cannula having a distal end with a marker exit disposed in the distal end; and a biopsy site marker, the marker delivery device being configured to expel the biopsy site marker through the marker exit and out of the needle through the upper opening defined by the tissue piercing tip.

### EXAMPLE 18

The biopsy system of Example 17, further comprising a first fluid source and a second fluid source, the first fluid source being configured to communicate vacuum or saline to the cutter lumen, the second fluid source being configured to communicate vacuum or saline to the lateral lumen.

### EXAMPLE 19

The biopsy system of Example 18, the first fluid source or the second fluid source being configured to heat saline to a temperature of between 35° C. to 40° C.

### EXAMPLE 20

The biopsy system of Examples 18 or 19, the lower opening being configured as a fluid outlet for the second fluid source, the upper opening being configured as a fluid inlet for the first fluid source.

### EXAMPLE 21

The biopsy system of Examples 18 or 19, the first fluid source being configured to communicate vacuum to the upper opening, the second fluid source being configured to communicate saline to the lower opening, the upper opening and the lower opening together being configured to define a saline path extending around an exterior of the tissue piercing tip from the lower opening to the upper opening.

### EXAMPLE 22

The biopsy system of any of Examples 17 through 21, the tissue piercing tip further defining a plurality of cutting surfaces, the upper opening and the lower opening each being disposed on a single cutting surface of the plurality of cutting surfaces.

### EXAMPLE 23

The biopsy system of any of Examples 17 through 22, the needle further including a lateral aperture and a cutter receiving tube, the upper opening being aligned with the cutter receiving tube, the lower opening being laterally offset relative to the cutter receiving tube and the lateral aperture.

### EXAMPLE 24

A method for marking a biopsy site comprising: positioning a biopsy needle proximate a biopsy site; collecting one or more tissue samples from the biopsy site by moving a cutter relative to the needle; inserting a marker delivery device into the needle through the cutter; expelling a marker from the marker delivery device towards the biopsy site through a marker lumen in a tissue piercing tip of the needle; and irrigating the biopsy site by communicating a fluid from a lower opening in the tissue piercing tip.

### EXAMPLE 25

The method of Example 24, further comprising extracting the fluid from the biopsy site by applying a vacuum to the marker lumen of the tissue piercing tip.

### EXAMPLE 26

The method of Example 25, the step of extracting the fluid being performed simultaneously with the step of irrigating the biopsy site.

### EXAMPLE 27

The method of any of Examples 24 through 26, further comprising warming the fluid prior to the step of irrigating the biopsy site.

### EXAMPLE 28

The method of any of Examples 24 through 27, the step of irrigating the biopsy site being performed before the step of inserting the marker delivery device into the needle.

### EXAMPLE 29

The method of any of Examples 24 through 28, the step of irrigating the biopsy site being performed before the step of collecting one or more tissue samples from the biopsy site.

### IV. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A needle for use with a biopsy device comprising:
(a) a cannula defining a cutter lumen configured to receive a cutter of the biopsy device, and a lateral lumen proximate the cutter lumen; and
(b) a tissue piercing tip including a body defining a plurality of cutting surfaces, a lower opening, and an upper opening, at least the upper opening being defined by a single cutting surface of the plurality of cutting surfaces, the upper opening being in communication with the cutter lumen, the lower opening being in communication with the lateral lumen.

2. The needle of claim 1, further comprising a lateral aperture, the lower opening being oriented away from a lateral aperture.

3. The needle of claim 2, the upper opening and the lateral aperture both being in communication with the cutter lumen.

4. The needle of claim 1, the upper opening being aligned with the cutter lumen.

5. The needle of claim 1, the body further defining an upper lumen and a lower lumen, the upper lumen being in communication with the upper opening, the lower lumen being in communication with the lower opening.

6. The needle of claim 5, the cutter lumen defining a cutter axis, the lateral lumen defining a lateral axis, the upper lumen being oriented at an angle with respect to the cutter axis, the lower lumen being aligned with the lateral axis.

7. The needle of claim 1, the cannula including an outer cannula and a cutter receiving tube, the outer cannula and the cutter receiving tube together defining at least a portion of the cutter lumen and the lateral lumen.

8. The needle of claim 7, the cutter receiving tube being disposed between the upper opening and the lower opening.

9. The needle of claim 1, the body further defining a plurality of cutting edges, the cutting edges together defining the plurality of cutting surfaces, each of the upper opening and the lower opening being disposed between at least two cutting edges.

10. The needle of claim 9, the plurality of cutting edges converging to form a sharp tip disposed distally of the upper opening and the lower opening.

11. The needle of claim 1, the upper opening being configured to receive a biopsy site marker.

12. The needle of claim 1, the tissue piercing tip being coupled to the cannula by a mechanical fastening mechanism.

13. The needle of claim 1, the upper opening defining a first shape, the lower opening defining a second shape, the first sample being different from the second shape.

14. The needle of claim 1, the upper opening defining a first diameter, the lower opening defining a second diameter, the first diameter being greater than the second diameter.

15. (canceled)

16. The needle of claim 1, the body further defining a first lower opening and a second lower opening, the first lower opening being oriented on an opposing side of the tissue piercing tip, the first lower opening and the second lower opening being offset with respect to the plurality of cutting surfaces.

17. A biopsy system comprising:
(a) a biopsy device including a body, a needle extending distally from the body, and a cutter movable relative to the needle, the needle including an outer cannula, a tissue piercing tip secured to a distal end of the outer cannula, a cutter lumen configured to receive the cutter, and a lateral lumen disposed proximate the cutter lumen, the tissue piercing tip defining a lower opening in communication with the lateral lumen and an upper opening in communication with the cutter lumen;
(b) a marker delivery device including an elongate cannula having a distal end with a marker exit disposed in the distal end; and
(c) a biopsy site marker, the marker delivery device being configured to expel the biopsy site marker through the marker exit and out of the needle through the upper opening defined by the tissue piercing tip.

18. The biopsy system of claim 17, further comprising a first fluid source and a second fluid source, the first fluid source being configured to communicate vacuum or saline to the cutter lumen, the second fluid source being configured to communicate vacuum or saline to the lateral lumen.

19.-20. (canceled)

21. The biopsy system of claim 18, the first fluid source being configured to communicate vacuum to the upper opening, the second fluid source being configured to communicate saline to the lower opening, the upper opening and the lower opening together being configured to define a saline path extending around an exterior of the tissue piercing tip from the lower opening to the upper opening.

22. The biopsy system of claim 17, the tissue piercing tip further defining a plurality of cutting surfaces, the upper opening and the lower opening each being disposed on a single cutting surface of the plurality of cutting surfaces.

23. (canceled)

24. A method for marking a biopsy site comprising:
(a) positioning a biopsy needle proximate a biopsy site;
(b) collecting one or more tissue samples from the biopsy site by moving a cutter relative to the needle;
(c) inserting a marker delivery device into the needle through the cutter;
(d) expelling a marker from the marker delivery device towards the biopsy site through a marker lumen in a tissue piercing tip of the needle; and
(e) irrigating the biopsy site by communicating a fluid from a lower opening in the tissue piercing tip.

25.-29. (canceled)
